(19) United States
(12) Patent Application Publication
(10) Pub. No.: US 2012/0022505 A1
(43) Pub. Date: Jan. 26, 2012

(54) Closed loop glaucoma drug delivery system

(76) Inventors: Bruno Dacquay, Irvine, CA (US); Robert J. Sanchez, JR., Oceanside, CA (US); Matthew J.A. Rickard, Yorba Linda, CA (US)

(21) Appl. No.: 13/109,155

(22) Filed: May 17, 2011

Related U.S. Application Data
(60) Provisional application No. 61/365,991, filed on Jul. 20, 2010.

(51) Int. Cl. A61M 5/00

(52) U.S. Cl. 604/891.1

(57) ABSTRACT

A device implantable into an eye of a patient for treatment of glaucoma. The device has an implantable sensor configured to measure at least one characteristic of the eye. The implantable sensor is sized for implantation into the eye. The device also has an implantable processor coupled to the sensor and configured to receive the measurement of the at least one characteristic of the eye and generate a signal based on the received measurement. Additionally, the device has an implantable actuator coupled to the processor and operable to release a dosage of a therapeutic agent into the eye in response to the signal from the implantable processor.

This application claims priority to U.S. Provisional Patent Application Ser. No. 61/365,991 filed on Jul. 20, 2010.

### BACKGROUND OF THE INVENTION

The present disclosure relates generally to an implantable variable drug system and more specifically to a closed loop implantable variable drug system used for treatment of glaucoma based on a characteristic of a patient's eye such as, for example, intraocular pressure (10P).

The eye's ciliary body epithelium constantly produces aqueous humor, the clear fluid that fills the anterior chamber of the eye (the space between the cornea and iris). Conventionally, the aqueous humor flows out of the anterior chamber through either the trabecular meshwork or the uveoscleral pathways. The delicate balance between the production and drainage of aqueous humor determines the eye's IOP.

Glaucoma, a group of eye diseases affecting the retina and optic nerve, is one of the leading causes of blindness worldwide. Glaucoma results when the IOP increases to pressures above normal for prolonged periods of time. IOP can increase due to increased resistance in the aqueous humor outflow path. Left untreated, an elevated IOP causes irreversible damage to the optic nerve and retinal fibers resulting in a progressive, permanent loss of vision.

Open angle (also called chronic open angle or primary open angle) is the most common type of glaucoma. With this type, even though the anterior structures of the eye appear normal, the resistance to aqueous flow builds downstream of the anterior chamber, either in the trabecular meshwork or further downstream (e.g. collector channels), causing the IOP to become elevated. Left untreated, this may result in permanent damage of the optic nerve and retina.

Healthcare professionals have found it difficult to obtain real time data on a continuous basis regarding a patient's IOP. Typically, a patient's IOP is checked by a healthcare provider during an office visit. However, a measured IOP for a given patient may be affected by the time of day, stress level of the patient, and other environmental and biological factors. Thus, the measured IOP value of patient during an office visit may not accurately reflect the average IOP of the patient throughout the day.

Moreover, the patient is typically prescribed the recommended drug dosage in the form of eye drops to treat lop abnormalities that may cause glaucoma. The patient is then left to self administer the eye drops. However, ensuring that the patient receives the prescribed dosage may be difficult because of the patient's inability to effectively deliver the drops into the eye and/or patient non-compliance by not using the eye drops altogether. Because frequent office visits to check IOP can be burdensome and time consuming, and because patient compliance can be variable, a need exists for an implantable variable drug system and more specifically for a closed loop implantable variable drug system used for treatment of glaucoma based on the IOP of a patient's eye.

The systems, devices, and methods disclosed herein overcome at least one of the shortcomings in the prior art.

### SUMMARY OF THE INVENTION

In one exemplary aspect, the present disclosure is directed to a device implanted into an eye of a patient for treatment of glaucoma. The device has an implantable sensor configured to measure at least one characteristic of the eye. The implantable sensor is sized for implantation in the eye. The device also has an implantable processor coupled to the sensor and configured to receive the measurement of the at least one characteristic of the eye and generate a signal based on the received measurement. Additionally, the device has an implantable actuator coupled to the processor and operable to release a dosage of a therapeutic agent into the eye in response to the signal from the implantable processor.

In one exemplary aspect, the present disclosure is directed to a method for delivering a therapeutic substance into an eye. The method includes implanting a system into the eye. The system includes an implantable sensor configured to measure at least one characteristic of the eye. The implantable sensor is sized for implantation into the eye and configured to detect at least one characteristic within the eye. The system also has an implantable processor coupled to the sensor and configured to receive the measurement of the at least one characteristic of the eye. Additionally, the system has an implantable actuator coupled to the processor and operable to release a dosage of a therapeutic agent into the eye in response to a signal from the implantable processor. The method further includes measuring the at least one characteristic of the eye with the implantable sensor. Also, the method includes determining a dosage of the therapeutic agent with the implantable processor. In addition, the method includes actuating the actuator to deliver the dosage into the eye in response to the signal from the implantable processor.

In one exemplary aspect, the present disclosure is directed to a device implantable into an eye of a patient for delivering a therapeutic agent. The device has a sensor configured to measure an intraocular pressure of the eye. Also, the device includes a processor coupled to the sensor and configured to receive the measured intraocular pressure. Additionally, the device has a memory coupled to the processor and configured to store the measured intraocular pressure. Also, the device has an actuator coupled to the processor and configured to release a dosage of a therapeutic agent into the eye.

These and other aspects, forms, objects, features, and benefits of the present disclosure will become apparent from the following detailed drawings and description.

### BRIEF DESCRIPTION OF THE DRAWINGS

The accompanying drawings, which are incorporated in and constitute a part of the specification, illustrate embodiments of the present disclosure. Together with a general description of the present disclosure given above, and the detailed description given below, the drawings serve to exemplify the embodiments of the present disclosure.

FIG. 1 is a block diagram of an exemplary implantable variable drug delivery system according to one aspect of the present disclosure.

FIG. 2 is a schematic diagram of an implantable portion of the implantable variable drug delivery system of FIG. 1.

FIG. 3 is an illustration of a perspective view of a patient's eye with the implantable portion of the variable drug delivery system of FIG. 2 implanted within the patient's eye.

FIG. 4 is a flow diagram showing exemplary steps for determining and administering a suitable drug dosage into the patient's eye using the implantable variable drug delivery system of FIG. 1.

### DETAILED DESCRIPTION OF THE INVENTION

The present disclosure relates generally to the field of ophthalmic surgery, and more particularly to an implantable variable drug system and more specifically to a closed loop implantable variable drug system used for treatment of glaucoma based on the IOP or other characteristic of a patient's eye. For the purposes of promoting an understanding of the principles of the present disclosure, reference will now be made to embodiments or examples illustrated in the drawings, and specific language will be used to describe these examples. It will nevertheless be understood that no limitation of the scope of the present disclosure is thereby intended. Any alteration and further modifications in the described embodiments, and any further applications of the principles of the present disclosure as described herein are contemplated as would normally occur to one skilled in the art to which the disclosure relates.

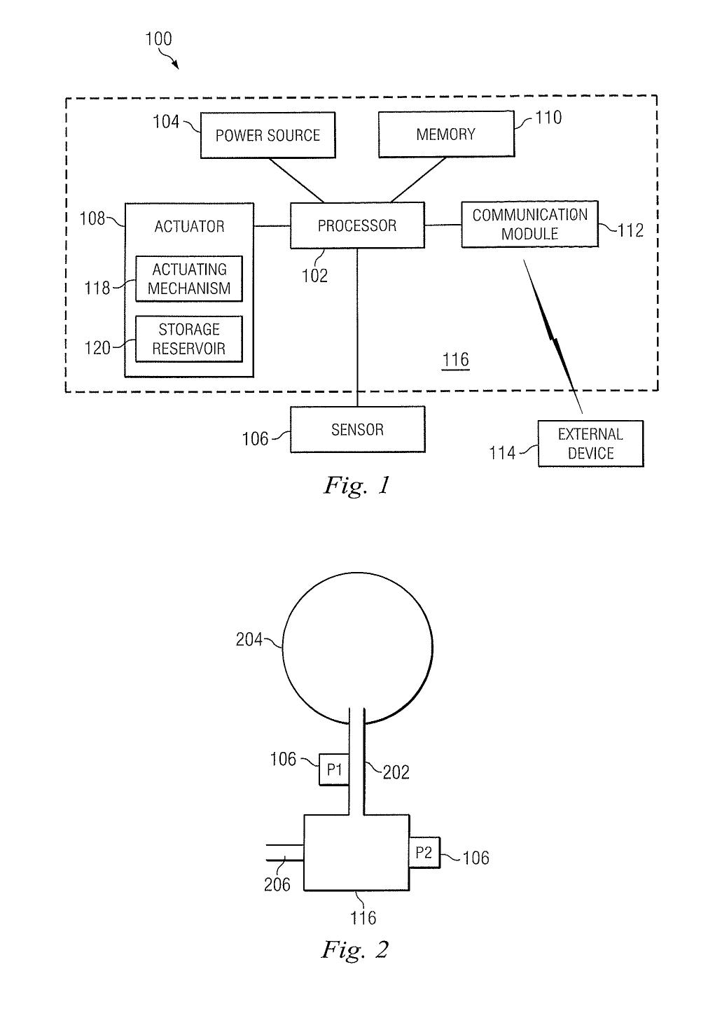

FIG. 1 is a schematic block diagram of an exemplary implantable variable drug delivery system according to one aspect of the present disclosure. The exemplary implantable drug delivery system 100 includes a processor 102, a power source 104, a sensor 106, an actuator 108, a memory 110, a communication module 112, and/or an external device 114.

Processor 102 controls the operating functions of the implantable system 100 and may be an integrated circuit with power, input, and output pins capable of performing logic functions. In various embodiments, processor 102 is a targeted device controller. In such a case, processor 102 performs specific control functions targeted to a specific device or component, such as power source 104, sensor 106, actuator 108, memory 110, and/or communication module 112.

In other embodiments, processor 102 is a microprocessor. In such a case, processor 102 is programmable so that it can function to control one or more of the components of system 100. In other cases, processor 102 is not a programmable microprocessor, but instead is a special purpose controller configured to control different components that perform different functions.

Power source 104 may be a rechargeable battery, such as a lithium ion or lithium polymer battery, although other types of batteries may be employed. Additionally, it is contemplated that power source 104 can be any type of power cell that is appropriate for implantation into the patient's body. In some embodiments, power source 104 is controllable by processor 102 to provide power to all the elements making up system 100. In other words, power source 104 may provide power to any component of system 100 including, but not limited to processor 102, sensor 106, actuator 108, memory 110, and/or communication module 112. In other embodiments, come components of system 100 have their own independent power source. In some examples, power source 102 is configured to be recharged via an RFID (radio-frequency identification) link or other type of magnetic coupling, or inductive coupling.

Sensor 106 is an IOP sensor configured to measure IOP in a patient's eye. Depending on the embodiment, sensor 106 includes one or more IOP sensors. For example, FIG. 2 shows a schematic diagram of an implantable portion of the implantable variable drug delivery system 100 of FIG. 1. In this example, the sensor 106 comprises a first pressure sensor P1 and a second pressure sensor P2. In that regard, pressure sensors P1 and P2 are shown attached to or incorporated into a housing 116. Housing 116 incorporates and/or encloses one or more of the components of system 100.

As shown in FIG. 2, a delivery tube 202 extends from the housing 116 and is in fluid communication with an anterior chamber 204 of a patient's eye. Pressure sensor P1 can be located either within anterior chamber 204 or in fluidic communication with the anterior chamber 204. In this example, pressure sensor P1 is positioned within or incorporated into delivery tube 202 such that the sensor P1 is in fluid communication with the anterior chamber 204. Thus, pressure sensor P1 is operable to measure a pressure in anterior chamber 204.

In other embodiments, pressure sensor P1 and any other component of system 100 including housing 116 and/or delivery tube 202 may be located in or partly in a subconjunctival space of the eye, a suprachoroidal space of the eye, a supraciliary space of the eye, a subscleral space of the eye, and/or outside the eye.

In some examples, pressure sensor P2 is positioned adjacent to or within the patient's eye and is operable to measure an atmospheric pressure. For example, pressure sensor P2 may be implanted in the eye under the conjunctiva (e.g. subconjunctival space), such that it measures atmospheric pressure. FIG. 2 shows pressure sensor P2 adjacent the housing 116, but it may also be positioned within or incorporated into housing 116. Regardless of location, pressure sensor P2 is operable to measure atmospheric pressure in the vicinity of the eye.

In another embodiment, P1 is positioned within the eye to measure the absolute pressure in the anterior chamber of the eye and P2 is located external to the eye and monitors/measures atmospheric pressure. For example, in such an embodiment P2 is located on an external device such as external device 114.

Here, processor 102 may utilize the readings from pressure sensors P1 and P2 to determine a patient's IOP. For purposes of this disclosure, IOP is measured as the difference between the absolute pressure in the eye (e.g. measurement taken by P1) and atmospheric pressure (e.g. measurement taken by P2). Pressure readings can be taken by P1 and P2 over any desired time interval. For example, in some embodiments, the pressure sensors P1 and P2 are programmed to continuously measure pressure, thereby providing real-time accuracy of the patient's IOP. In other embodiments, pressure readings are taken by P1 and P2 at pre-established time intervals. For example, readings may be taken every minute, hourly, daily, etc. Regardless of the frequency of the pressure readings, the patient's IOP can be calculated based on the difference between the pressure readings of P1 and P2.

As described above, the pressure readings of P1 and P2 can be used to calculate the patient's current IOP. They can also be used to calculate the patient's average IOP over a given time period. For example, the pressure readings of P1 and P2 can be used to calculate the patient's IOP for a given time of day and/or week. In other words, it is contemplated that system 100 can use the pressure readings of P1 and P2 to determine the patient's IOP based on any desired interval.

It is contemplated that pressure sensors P1 and P2 can be any type of pressure sensor suitable for implantation in the eye. Furthermore, pressure sensors P1 and P2 can be the same type of pressure sensor, or may be different types. Moreover, although sensor 106 has been discussed as comprising two pressure sensors (e.g. P1 and P2) it is contemplated that a patient's IOP may be determined by using a single pressure sensor or through using three or more pressure sensors. Accordingly, no limitation to the number of or type of pressure sensors is implied by the present disclosure.

Returning to FIG. 1, also coupled to processor 102 is actuator 108. Actuator 108 represents a therapeutic, or drug, delivery module that has an actuating mechanism 118 and a storage reservoir 120 for administering and storing a drug. In that regard, system 100 provides the ability to monitor a patient's IOP via pressure readings P1 and P2 and subsequently administer a therapeutic agent that controls IOP. The actuating mechanism 118 of actuator 108 can utilize, for example, delivery tube 202 (FIG. 2) to deliver therapeutic agents into anterior chamber 204 of a patient's eye. The therapeutic agent may be, for example, a solid, liquid, granule, and/or soluble agent designed to control a patient's IOP. Thus, actuator 108 is operable to deliver a therapeutic agent into a patient's eye to control a patient's IOP.

Furthermore, as shown in FIG. 2, housing 116 can have an inlet port 206 that is in fluid communication with the storage reservoir 120 of the actuator 108. Inlet port 206 enables in vivo filling and/or refilling of a therapeutic agent for system 100. It is further contemplated that the storage reservoir can be refilled with a therapeutic agent that is either substantially the same and/or substantially different than the previous therapeutic agent.

In one example, the storage reservoir 120 is compartmentalized such that more than one therapeutic agent can be stored in two or more compartments of the reservoir. As such, system 100 may be configured to deliver two or more different therapeutic agents. System 100 may control a patient's IOP by controlling the rate of release for these two or more therapeutic agents. For example, system 100 utilizes actuating mechanism 118 to release a first therapeutic agent at a first rate and a second therapeutic agent at a second rate that is different than the first. In another example, system 100 controls the release of a first therapeutic agent that decreases a patient's IOP at a first rate of change and a second therapeutic agent that decreases a patient's IOP at a second rate of change that is different than the first. In other embodiments, system 100 controls a patient's IOP by controlling the release rate for one or more therapeutic agents as well as using one or more therapeutic agents that decrease a patient's IOP at various rates of change.

Referring to FIG. 1, memory 110 is any type of suitable storage memory including, but not limited to flash memory, solid state memory, organic memory, inorganic memory, and others. Memory 110 interfaces with processor 102. Thus, processor 102 can write to and read data from memory 110.

In some examples, memory 110 is operable to store dosage parameters or logic such as executable code. In that regard, memory 110 can store programming data (e.g. dosage parameters) accessible by processor 102 that enables the processor to determine the proper dosage to deliver to a patient. In other words, based on the data and code stored in memory 110, processor 102 determines the proper dosage for a patient and subsequently actuates actuator 108 to deliver the drug to the patient. In some examples, processor 102 is hard coded or programmed directly with such dosage parameters such the processor can determine the proper dosage without accessing memory 110.

The memory 110 is also configured to store pressure readings of P1 and P2. For example, processor 102 receives data from the IOP sensor 106 and subsequently writes the data to memory 110. In this manner, a series of IOP readings can be stored in memory 110. Processor 102 is also capable of performing other basic memory functions, such as erasing or overwriting memory 110, detecting when memory 110 is full, and other common functions associated with managing memory.

The communication module 112 in FIG. 1 is operable to transmit and receive a number of different types of data transmission, or signals to external systems. For example, as shown in FIG. 1, communication module 112 can communicate with external device 114. Communication module 112 is operable to transmit and/or receive any data relating to system 100. For example, communication module 112 is operable to transmit and receive data relating to the measured pressure readings from sensor 106, patient's calculated IOP, dosage parameters, and/or any other data collected by system 100. The therapeutic dosage parameters may include, but not limited to the factors that determine the frequency and amount of therapeutic agent to be delivered to a patient.

In one example, communication module 112 is an active communication module such as a radio. As an active communication module, data collected by system 100 is actively transmitted to external device 114 positioned external of the patient. In other embodiments, communication module 112 is a passive module. For example, communication module 112 may be a passive RFID device. As such, communication module 112 is operable to transmit and receive data when activated by radio frequency signals to the external device 114.

As discussed above, communication module 112 is operable to transmit and receive data to and from external device 114. For example, external device 114 may include, but not limited to a computer system particularly arranged to communicate with system 100, PDA, cell phone, wrist watch, custom device exclusively for this purpose, remote accessible data storage site (e.g. an internet server, email server, text message server), or other electronic device. As such, these external devices allow a healthcare professional to monitor and treat a patient's IOP.

For example, the healthcare professional can receive data relating to a patient's IOP from communication module 112 on external device 114 (e.g. computer). Based upon the received data, the healthcare provider can diagnose and determine whether the dosage parameters stored in system 100 adequately address the patient's needs. If the healthcare provider determines that the dosage parameters need altering or updating, then the healthcare provider can interface with system 100 through communication module 112. In such a scenario, the healthcare provider can alter or update the dosage parameters stored in processor 102 and/or memory 110 via their external device 114 (e.g. computer). Thus, communication module 112 enables the healthcare provider to have an accurate accounting of the patient's IOP condition as well as the ability to alter the course of treatment if needed.

In some embodiments communication module 112 is operable to receive data transmissions/signals that can be used to charge power source 104. In other words, signals received by communication module 112 can be used to provide energy to system 100, including the ability to charge power source 104. In some examples, communication module 112 includes an antenna capable of harvesting energy through inductive coupling with one of the external devices discussed above. In that regard, communication module 112 can harvest energy from signals, such as radio frequency waves, in order to provide power to system 100.

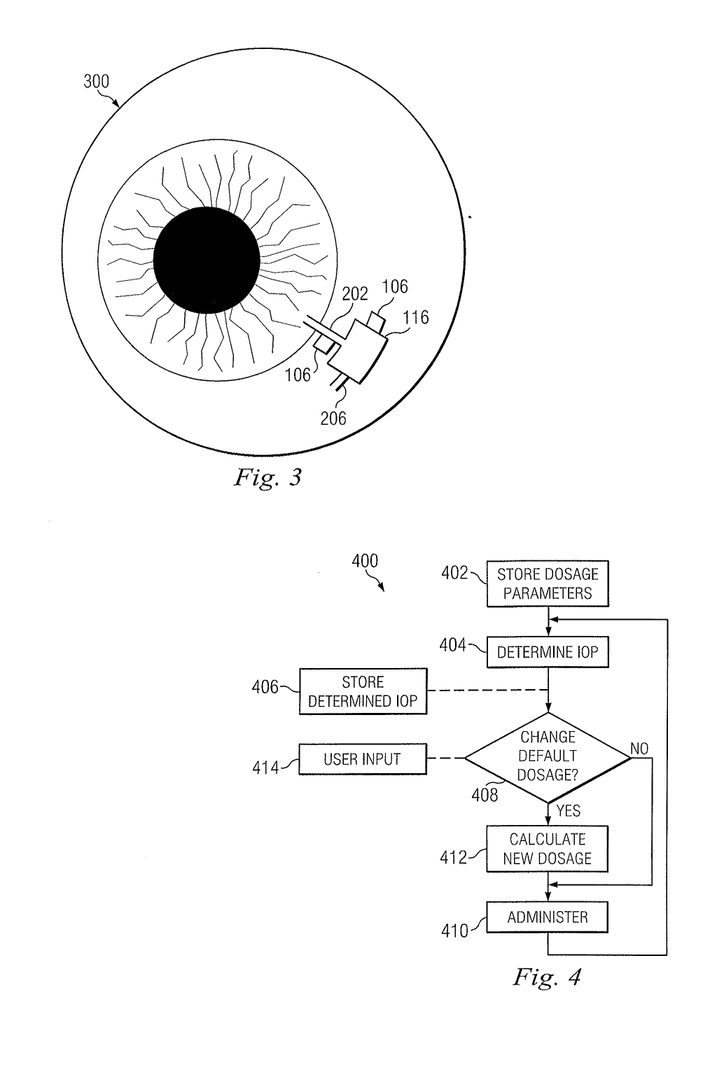

FIG. 3 is an illustration of a perspective view of a patient's eye with the implantable portion of the variable drug delivery system 100 implanted therein. Here, at least some components of system 100 are housed within housing 116 which is implanted under the conjunctiva of eye 300. In other embodiments, however, housing 116 including one or more of the components of system 100 is implanted on the exterior of the sclera of eye 300. Moreover, it is contemplated that housing 116 and/or one or more of the components of system 100 can be implanted anywhere within the eye to provide the ability to monitor and treat a patient's IOP. In other embodiments, one or more of the components of system 100 are located in or partly in a subconjunctival space of the eye, a suprachoroidal space of the eye, a supraciliary space of the eye, a subscleral space of the eye, and/or outside the eye.

FIG. 4 is an exemplary flow diagram showing a method 400 for determining the drug dosage delivered into the patient's eye using the implantable variable drug delivery system 100. Method 400 begins at step 402 with a step of storing dosage parameters in memory 110 and/or processor 102 of system 100. The dosage parameters represent the logic used by system 100 to determine the dosage to administer to the patient to control 10P. The dosage parameters can be stored in memory 110 and/or processor 102 prior to, during, or after implantation of system 100 within the patient's eye. In this regard, the dosage parameters represent programming logic that allows processor 102 to determine the frequency, amount, and/or which therapeutic agent to administer to a patient.

In some aspects, the dosage parameter includes a default dosage. The default dosage represents a dosage of a therapeutic agent that is administered to the patient as established by the healthcare provider without accounting for data accumulated by system 100 (e.g. measured IOP by sensor 106). Thus, in some embodiments, system 100 can be implemented to administer a default dosage regimen that is not altered after being stored in system 100 regardless of the collected data.

Step 404 represents processor 102 receiving pressure readings from P1 and P2 of sensor 106, and based upon the readings, the processor 102 subsequently determines the patient's IOP. As discussed above, some embodiments of the system store the pressure readings from P1 and P2 of sensor 106. The dashed line at step 406 represents the optional nature of storing the pressure readings of sensor 106 in memory 110.

The operation of system 100 continues to step 408 where the system determines whether to change the default dosage. As discussed above, the default dosage can be administered to the patient without accounting for and/or considering the data accumulated by system 100 (e.g. measured IOP). If system 100 has been programmed as such, then the system administers the default dosage at step 410.

However at step 408, if the dosage parameters have been programmed to account for data accumulated by the system (e.g. measured IOP), then processor 102 determines the dosage to administer to the patient based on the collected data. In response to the collected data, processor 102 may change the default dosage.

For example, accessing the dosage parameters, processor 102 can be programmed to compare the patient's measured IOP against an acceptable range for the patient's IOP as set forth in the dosage parameters. If the patient's measured IOP falls outside of the acceptable range of IOP, then processor 102 may change the default dosage. If however, the patient's measured IOP falls within the acceptable range of IOP, then the processor may not change the default dosage and subsequently administer the default dosage at step 410.

If the default dosage should be changed at step 408, then at step 412, processor 102 calculates a new dosage (e.g. change the default dosage). Again, the processor 102 may rely upon dosage parameters stored in system 100 for determining a new frequency, amount, and/or type of therapeutic agent to administer to the patient. After the new dosage has been calculated, system 100 administers the new dosage at step 410.

In some examples, the step of determining whether to change the default dosage at step 408 includes considering a user input. The dashed line at step 414 represents the optional nature of considering user input. As discussed above, a healthcare provider can interface with system 100 via external device 114. As such, the healthcare provider can alter or update the stored dosage parameters via the communication module 112. Thus, the healthcare provider can instruct system 100 to change the default dosage thereby altering the patient's course of treatment.

Upon administering the default dosage or new dosage at step 410, the method of operation for system 100 returns to step 404. As such, the system continues to monitor and measure the patient's IOP until the IOP or other parameters dictate that the system administer another dosage of a therapeutic agent.

In summary, implantable system 100 allows for the monitoring and treating excessive fluctuations of a patient's IOP. Unlike traditional treatments for IOP, patient compliance is a non-issue because the implantable system 100 automatically delivers the therapeutic agent at the appropriate dosage. Moreover, because system 100 has the ability to continuously monitor and store IOP data, the system allows a healthcare provider to access and download a complete overview of the patient's IOP for a given time period. The doctor then has the ability to review this extensive IOP data in order to make a more accurate decision regarding future care of the patient (e.g. alter dosage parameters).

In addition, system 100 allows a healthcare provider to set a default dosage to administer the therapeutic agent to treat the patient's IOP. In that regard, system 100 is operable to release the drug at the default dosage without accounting for data accumulated by system 100 (e.g. measured IOP). Additionally, as discussed above, the system 100 can release the therapeutic agent as determined by a closed loop feedback control system based on IOP. Particularly, system 100 can release the therapeutic agent at a default dosage rate initially for a predetermined amount of time and then can change the dosage amount over to a closed loop control method in which the system uses a closed loop feedback based on IOP measurements to adjust the dosage. Additionally, it is contemplated that system 100 may also have a high and low dosage limit to prevent an over-dosage or under-dosage of a therapeutic agent.

While the present disclosure has been illustrated by the above description of embodiments, and while the embodiments have been described in some detail, it is not the intention of the applicant to restrict or in any way limit the scope of the present disclosure to such detail. Additional advantages and modifications will readily appear to those skilled in the art. Therefore, the present disclosure in its broader aspects is not limited to the specific details, representative apparatus and methods, and illustrative examples shown and described. Accordingly, departures may be made from such details without departing from the spirit or scope of the applicant's general or inventive concept.

We claim:

1. A device implanted into an eye of a patient for treatment of glaucoma, the device comprising:
an implantable sensor configured to measure at least one characteristic of the eye, the implantable sensor being sized for implantation into the eye;
an implantable processor coupled to the sensor and configured to receive the measurement of the at least one characteristic of the eye and generate a signal based on the received measurement; and
an implantable actuator coupled to the processor and operable to release a dosage of a therapeutic agent into the eye in response to the signal from the implantable processor.

2. The device of claim 1, wherein the implantable processor determines the dosage based upon the at least one characteristic of the eye.

3. The device of claim 1, wherein the at least one characteristic of the eye is an intraocular pressure of the eye.

4. The device of claim 1, wherein the implantable sensor is comprised of first and second pressure sensors, the first pressure sensor being in fluid communication with an anterior chamber of the eye in order to measure a first pressure of the anterior chamber and the second pressure sensor being in fluid communication with an atmosphere adjacent the eye in order to measure a second pressure of the atmosphere.

5. The device of claim 4, wherein the implantable processor is operable to receive the measured first and second pressures to determine the intraocular pressure of the eye of the patient.

6. The device of claim 1, further comprising a delivery tube operable to deliver the therapeutic agent into an anterior chamber of the eye upon actuation of the actuator.

7. The device of claim 6, wherein at least a portion of the sensor is coupled with the delivery tube.

8. A method for delivering a dosage of a therapeutic substance into an eye, the method comprising:
implanting a system into the eye, the system comprising:
an implantable sensor configured to measure at least one characteristic of the eye, the implantable sensor being sized for implantation into the eye and configured to detect the at least one characteristic within the eye
an implantable processor coupled to the sensor and configured to receive the measurement of the at least one characteristic of the eye; and
an implantable actuator coupled to the processor and operable to release a dosage of a therapeutic agent into the eye in response to a signal from the implantable processor; and
measuring the at least one characteristic of the eye with the implantable sensor;
determining the dosage of the therapeutic agent with the implantable processor; and
actuating the actuator to deliver the dosage into the eye in response to the signal from the implantable processor.

9. The method of claim 8, wherein the at least one characteristic is an intraocular pressure of the eye.

10. The method of claim 8, wherein determining the dosage includes utilizing the received measurement of the at least one characteristic of the eye to determine the dosage.

11. The method of claim 8, wherein the system further comprises a delivery tube coupled to the actuator,
wherein implanting the system includes implanting at least a portion of the delivery tube into an anterior chamber of the eye, and
wherein delivering the dosage includes delivering the therapeutic agent through the delivery tube into the anterior chamber of the eye.

12. The method of claim 8, wherein the sensor is comprised of first and second pressure sensors for measuring the at least one characteristic of the eye;
wherein measuring the at least one characteristic of the eye includes the first pressure sensor measuring a first pressure of an anterior chamber of the eye and the second pressure sensor measuring an atmospheric pressure; and
wherein determining the dosage for the therapeutic agent includes the processor determining an intraocular pressure of the eye using the first and second pressures and determining the dosage based on the intraocular pressure.

13. The method of claim 8, further comprising refilling the system in vivo with the therapeutic agent.

14. A device implantable into an eye of a patient for delivering a therapeutic agent, the device comprising:
a sensor configured to measure an intraocular pressure of the eye;
a processor coupled to the sensor and configured to receive the measured intraocular pressure;
a memory coupled to the processor and configured to store the measured intraocular pressure; and
an actuator coupled to the processor and configured to release a dosage of a therapeutic agent into the eye.

15. The device of claim 14, wherein the actuator has a reservoir for storing the therapeutic agent.

16. The device of claim 15, wherein the reservoir has at least two compartments and the therapeutic agent is two or more therapeutic agents stored separately in the at least two compartments, and
wherein the actuator is operable to separately release the two or more therapeutic agents.

17. The device of claim 15, wherein the actuator has a port in fluid communication with the reservoir such that the reservoir is refillable in vivo with a second therapeutic agent.

18. The device of claim 17, wherein the second therapeutic agent is different than the therapeutic agent.

19. The device of claim 14, further comprising a communication module coupled to the processor, the communication module operable to receive a signal from an external source that defines the dosage.

20. The device of claim 19, further comprising a power source coupled to the processor, wherein the processor is operable to use the signal from the external source to provide energy to the power source.
